(19) United States
(12) Patent Application Publication
(10) Pub. No.: US 2005/0106275 A1
(43) Pub. Date: May 19, 2005

(54) Morinda citrifolia-based formulation for inhibiting metastasis of carcinogenic cells

(76) Inventors: Chen Su, West Jordan, UT (US); Brett Justin West, Orem, UT (US); Afa Kehaati Palo, Orem, UT (US); Anne Hirasumi-Kim, Mililani, HI (US); Claude Jarakae Jensen, Cedar Hills, UT (US); Stephen P. Story, Apline, UT (US)

Correspondence Address: KIRTON & McCONKIE, 60 East South Temple, Suite 1800, Salt Lake City, UT, 84111, US

(21) Appl. No.: 10/836,571

(22) Filed: May 1, 2004

Related U.S. Application Data
(60) Provisional application No. 60/467,723, filed on May 2, 2003.

(51) Int. Cl. A61K 35/78

(52) U.S. Cl. 424/769; 424/777

(57) ABSTRACT

The present invention features processed ingredients from the Indian mulberry plant, and particularly Morinda citrifolia fruit juice, for inhibiting and preventing metastasis of carcinogenic cells, as well as destroying metastasized cells. At least some implementations of the present invention comprise the consumption of food products or medicinal products or compositions comprising processed Morinda citrifolia.

### RELATED APPLICATIONS

This application claims priority to U.S. Patent Application Ser. No. 60/467,723 filed May 2, 2003, entitled MORINDA CITRIFOLIA-BASED FORMULATION AND METHOD FOR TREATING METASTASIS OF CARCINOGENIC CELLS.

### BACKGROUND OF THE INVENTION

1. Field of the Invention

The present invention relates to inhibiting metastasis of carcinogenic cells. In particular, the present invention relates to medicinal products, as well as health and well-being food products, and particularly to a medicinal product or a health and well-being food product designed to inhibit, block, and/or prevent metastasis of carcinogenic cells away from a primary cancer site.

2. Background and Related Art

Metastasis is the spread or movement of cancer cells from the primary cancer site to another area of the body. More particularly, metastasis is the movement or spreading of malignant cancer cells from their original site to distant organs. Other than certain white blood cells, this is something most normal cells cannot do, and it is the most deadly characteristic of cancer. In spite of a large amount of research focused in this area, there are very few drugs or therapeutic treatments available for patients with metastatic cancer disease and searching for new entities to conquer this deadly disease has been an urgent task for decades for both research scientists and medical doctors. Over 50% of cancer patients die from metastasis.

During metastasis, tumor cells penetrate the fibrous boundaries that normally separate one tissue from another. The tumor can also infiltrate the walls of blood or lymph vessels and shed cancer cells into the circulation. In the blood, these tumor cells are carried downstream to become lodged in the next capillary bed. Tumor cells shed from colon cancer, for example, are carried by the circulation to the liver, where secondary tumors then arise. Tumor cells from other areas of the body can be carried by the blood through the heart and on to the lungs, where they start metastatic lung tumors. Tumor cells shed into the lymph system often establish themselves in the nearest cluster of lymph nodes, where they grow before spreading to more distant parts of the body. Fewer than 1 in 10,000 cells shed from the primary tumor are thought to survive, but these are enough to spawn secondary tumors elsewhere in the body.

About 30 percent of new patients with solid tumors have detectable metastases. About half the remaining patients will be cured by treating the tumor alone. The remainder will have undetectable metastases that will eventually develop into tumors. Tumor staging includes a measure of whether a malignancy has spread beyond the primary tumor. This is a major factor in determining a patient's prognosis.

The goal of early detection is to remove the primary tumor before metastasis has occurred. Unfortunately, some tumors apparently metastasize before they are large enough to be found. The spread of such micro-metastases may explain why many women die of breast cancer even after early detection of their primary tumors.

Thus, metastasis is the movement or spreading of malignant cancer cells from their original site to distant organs. Over 50% of cancer patients die from metastasis. In spite of a large amount of research focused in this area, there are very few drugs or therapeutic treatments available for patients with metastatic cancer disease and searching for new entities to conquer this deadly disease has been an urgent task for decades for both research scientists and medical doctors. Accordingly, it would be an improvement in the art to effectively inhibit metastasis.

### SUMMARY OF THE INVENTION

The present invention relates to inhibiting metastasis of carcinogenic cells. In particular, the present invention relates to medicinal products, as well as health and well-being food products, and particularly to a medicinal product or a health and well-being food product designed to inhibit, block, and/or prevent metastasis of carcinogenic cells away from a primary cancer site.

Implementation of the present invention takes place in association with a formulation comprising one or more forms of processed Morinda citrifolia for treating metastasis of carcinogenic cells, and particularly to the inhibition, blocking, and/or prevention of metastasis using. At least some implementations of the present invention includes a naturaceutical formulation for treating metastasis, wherein the formulation comprises one or more processed Morinda citrifolia products present in an amount between about 0.01 and 100 percent by weight. In some implementations, the Morinda citrifolia product comprises Morinda citrifolia fruit juice or fruit juice concentrate. In other implementations, the Morinda citrifolia product comprises puree juice or puree concentrate. Also, a combination of these together and/or mixed with other natural ingredients is also contemplated.

The present invention further features a method for inhibiting and preventing metastasis of carcinogenic cells, as well as destroying early stage metastasized carcinogenic cells. The method comprises the steps of adding a processed Morinda citrifolia product to an alcohol-based solution, isolating and extracting an active ingredient of Morinda citrifolia from the solution, introducing the extracted active ingredient to an area afflicted by carcinogenic cells, wherein the extracted active ingredient inhibits and prevents further growth of carcinogenic cells, as well as destroys early stage metastasized carcinogenic cells.

At least some implementations relate to an inhibition of tumor metastasis of an ethanol insoluble precipitate from Morinda citrifolia L fruit juice. An ethanol insoluble precipitate (polysaccharides) from Morinda citrifolia L (“Morinda citrifolia-PPT”) is used to provide anti-metastatic activity in the syngeneic tumor metastasis model of melanoma B16-F0 cells. Research in mice resulted in the Morinda citrifolia-PPT showing a 22% inhibitory effect (p<0.005) at 0.8 mg per mouse.

GPC (gel permeation chromatography) analysis demonstrated that the Morinda citrifolia-PPT includes four different molecular weight molecules, at 879, 2045, 14,461, and 84,076 Daltons, respectively. Using anion exchange chromatography, the fraction with MW 84,076 was derived by eluting Morinda citrifolia-PPT with 0.1 N NaCl. Morinda citrifolia-PPT and the fraction with MW 84,076 showed 36% and 37% adhesion inhibition at 10 mg/ml and 1 mg/ml, respectively, of NRK 2 cells to a fibronectin-coated well.

Although the inhibition of fibronectin adhesion can be one of the mechanisms for Morinda citrifolia-PPT's anti-metastatic effect, the relatively weak adhesion effect in the fibronectin mediated adhesion experiment suggests that there may be more than one mechanism involved. The carbohydrate content analysis indicated that Morinda citrifolia-PPT contained 70% galacturonic acid, determined by UV spectrometer. This finding implied that Morinda citrifolia-PPT suppresses the adhesion of melanoma B16-F0 to the lung tissue by binding to a lung endothelial surface molecule, Lu-ECAM-1-a galacoside-binding protein. It also suggests that Morinda citrifolia-PPT exerts an anti-metastasis effect through blocking cell-cell and cell-substrate binding for many malignant cell surface expressed galectin-galactoside binding proteins.

In at least some of the research, Morinda citrifolia-PPT did not exhibit any inhibition effect of endothelial cell tube formation. Accordingly, Morinda citrifolia-PPT's anti-metastatic effect is acting as an anti-adhesive agent to inhibit the formation of tumor cell emboli and the tumor cell-cell interaction.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### BRIEF DESCRIPTION OF THE DRAWINGS

In order that the manner in which the above recited and other features and advantages of the present invention are obtained, a more particular description of the invention will be rendered by reference to specific embodiments thereof, which are illustrated in the appended drawings. Understanding that the drawings depict only typical embodiments of the present invention and are not, therefore, to be considered as limiting the scope of the invention, the present invention will be described and explained with additional specificity and detail through the use of the accompanying drawings in which:

FIG. 1A depicts chromatography chart for Morinda citrifolia-PPT GPC analysis.

FIG. 1B depicts chromatography chart for 0.1 M NaCl fraction GPC analysis.

FIG. 2 depicts photographs of tube formation for Morinda citrifolia-PPT, vehicle, and positive control, pacitaxel.

### DETAILED DESCRIPTION OF THE INVENTION

The present invention relates to preventing metastasis of carcinogenic cells. In particular, the present invention relates to medicinal products, as well as health and well-being food products, and particularly to a medicinal product or a health and well-being food product designed to inhibit, block, and/or prevent metastasis of carcinogenic cells away from a primary cancer site.

The following disclosure of the present invention is grouped into two subheadings, namely “General Description of Morinda citrifolia” and “Inhibiting Metastasis.” The utilization of the subheadings is for convenience of the reader only and is not to be construed as limiting in any sense.

### General Description of Morinda Citrifolia

Embodiments of the present invention include a formulation comprising one or more forms of processed Morinda citrifolia for treating metastasis of carcinogenic cells, and particularly to the inhibition, blocking, and/or prevention of metastasis using. Accordingly, the following is a general description of Morinda citrifolia, including its origins, processing techniques, and health benefits. A more detailed description of the Morinda citrifolia-based formulations and compositions used to treat metastasis and the methods used for administering these to a subject, including examples of experimental studies and the results attained, is provided below.

The Indian Mulberry or Morinda citrifolia plant, known scientifically as Morinda Citrifolia L. (“Morinda citrifolia”), is a shrub or small tree up to 10 m in height. The leaves are oppositely arranged with an elliptic to ovate form. The small white flowers are contained in a fleshy, globose, head-like cluster. The fruits are large, fleshy, and ovoid. At maturity, they are creamy-white and edible, but have an unpleasant taste and odor. The plant is native to Southeast Asia and has spread in early times to a vast area from India to eastern Polynesia. It grows randomly in the wild, and it has been cultivated in plantations and small individual growing plots. The Morinda citrifolia flowers are small, white, three to five lobed, tubular, fragrant, and about 1.25 cm long. The flowers develop into compound fruits composed of many small drupes fused into an ovoid, ellipsoid or roundish, lumpy body, with waxy, white, or greenish-white or yellowish, semi-translucent skin. The fruit contains “eyes” on its surface, similar to a potato. The fruit is juicy, bitter, dull-yellow or yellowish-white, and contains numerous red-brown, hard, oblong-triangular, winged 2-celled stones, each containing four seeds.

When fully ripe, the fruit has a pronounced odor like rancid cheese. Although the fruit has been eaten by several nationalities as food, the most common use of the Morinda citrifolia plant was as a red and yellow dye source. Recently, there has been an interest in the nutritional and health benefits of the Morinda citrifolia plant, further discussed below.

Because the Morinda citrifolia fruit is for all practical purposes inedible, the fruit must be processed in order to make it palatable for human consumption and included in food products used to treat Candidiasis. Processed Morinda citrifolia fruit juice can be prepared by separating seeds and peels from the juice and pulp of a ripened Morinda citrifolia fruit; filtering the pulp from the juice; and packaging the juice. Alternatively, rather than packaging the juice, the juice can be immediately included as an ingredient in another food product, frozen or pasteurized. In some embodiments, the juice and pulp can be pureed into a homogenous blend to be mixed with other ingredients. Other process include freeze drying the fruit and juice. The fruit and juice can be reconstituted during production of the final juice product. Still other processes include air drying the fruit and juices, prior to being masticated.

The present invention utilizes the fruit juice and the oil extracted from the Morinda Citrifolia plant. In a currently preferred process of producing Morinda citrifolia fruit juice, the fruit is either hand picked or picked by mechanical equipment. The fruit can be harvested when it is at least one inch (2-3 cm) and up to 12 inches (24-36 cm) in diameter. The fruit preferably has a color ranging from a dark green through a yellow-green up to a white color, and gradations of color in between. The fruit is thoroughly cleaned after harvesting and before any processing occurs.

The fruit is allowed to ripen or age from 0 to 14 days, with most fruit being held from 2 to 3 days. The fruit is ripened or aged by being placed on equipment so it does not contact the ground. It is preferably covered with a cloth or netting material during aging, but can be aged without being covered. When ready for further processing the fruit is light in color, from a light green, light yellow, white or translucent color. The fruit is inspected for spoilage or for excessively green color and hard firmness. Spoiled and hard green fruit is separated from the acceptable fruit.

The ripened and aged fruit is preferably placed in plastic lined containers for further processing and transport. The containers of aged fruit can be held from 0 to 30 days. Most fruit containers are held for 7 to 14 days before processing. The containers can optionally be stored under refrigerated conditions prior to further processing. The fruit is unpacked from the storage containers and is processed through a manual or mechanical separator. The seeds and peel are separated from the juice and pulp.

The juice and pulp can be packaged into containers for storage and transport. Alternatively, the juice and pulp can be immediately processed into finished juice product. The containers can be stored in refrigerated, frozen, or room temperature conditions. The Morinda citrifolia juice and puree are preferably blended in a homogenous blend, after which they may be mixed with other ingredients, such as flavorings, sweeteners, nutritional ingredients, botanicals, and colorings. The finished juice product is preferably heated and pasteurized at a minimum temperature of 181° F. (83° C.) or higher up to 212° F. (100° C.).

The product is filled and sealed into a final container of plastic, glass, or another suitable material that can withstand the processing temperatures. The containers are maintained at the filling temperature or may be cooled rapidly and then placed in a shipping container. The shipping containers are preferably wrapped with a material and in a manner to maintain or control the temperature of the product in the final containers.

The juice and pulp may be further processed by separating the pulp from the juice through filtering equipment. The filtering equipment preferably consists of, but is not limited to, a centrifuge decanter, a screen filter with a size from 1 micron up to 2000 microns, more preferably less than 500 microns, a filter press, reverse osmosis filtration., and any other standard commercial filtration devices. The operating filter pressure preferably ranges from 0.1 psig up to about 1000 psig. The flow rate preferably ranges from 0.1 g.p.m. up to 1000 g.p.m., and more preferably between 5 and 50 g.p.m. The wet pulp is washed and filtered at least once and up to 10 times to remove any juice from the pulp. The wet pulp typically has a fiber content of 10 to 40 percent by weight. The wet pulp is preferably pasteurized at a temperature of 181° F. (83° C.) minimum and then packed in drums for further processing or made into a high fiber product.

The method for extracting and processing the oil is described in co-pending application Ser. No. 09/384,785, filed on Aug. 27, 1999, which is incorporated by reference herein. The Morinda citrifolia oil typically includes a mixture of several different fatty acids as triglycerides, such as palmitic, stearic, oleic, and linoleic fatty acids, and other fatty acids present in lesser quantities. In addition, the oil preferably includes an antioxidant to inhibit spoilage of the oil. Conventional food grade antioxidants are preferably used.

The Morinda citrifolia plant is rich in natural ingredients. Those ingredients that have been discovered include: from the leaves: alanine, anthraquinones, arginine, ascorbic acid, aspartic acid, calcium, beta-carotene, cysteine, cystine, glycine, glutamic acid, glycosides, histidine, iron, leucine, isoleucine, methionine, niacin, phenylalanine, phosphorus, proline, resins, riboflavin, serine, beta-sitosterol, thiamine, threonine, tryptophan, tyrosine, ursolic acid, and valine; from the flowers: acacetin-7-o-beta-d (+)-glucopyranoside, 5,7-dimethyl-apigenin-4′-o-beta-d(+)-galactopyranoside, and 6,8-dimethoxy-3-methylanthraquinone-1-o-beta-rhamnosyl-glucopyranoside; from the fruit: acetic acid, asperuloside, butanoic acid, benzoic acid, benzyl alcohol, 1-butanol, caprylic acid, decanoic acid, (E)-6-dodeceno-gamma-lactone, (Z,Z,Z)-8,11,14-eicosatrienoic acid, elaidic acid, ethyl decanoate, ethyl hexanoate, ethyl octanoate, ethyl palmitate, (Z)-6-(ethylthiomethyl) benzene, eugenol, glucose, heptanoic acid, 2-heptanone, hexanal, hexanamide, hexanedioic acid, hexanoic acid (hexoic acid), 1-hexanol, 3-hydroxy-2-butanone, lauric acid, limonene, linoleic acid, 2-methylbutanoic acid, 3-methyl-2-buten-1-ol, 3-methyl-3-buten-1-ol, methyl decanoate, methyl elaidate, methyl hexanoate, methyl 3-methylthio-propanoate, methyl octanoate, methyl oleate, methyl palmitate, 2-methylpropanoic acid, 3-methylthiopropanoic acid, myristic acid, nonanoic acid, octanoic acid (octoic acid), oleic acid, palmitic acid, potassium, scopoletin, undecanoic acid, (Z,Z)-2,5-undecadien-1-ol, and vomifol; from the roots: anthraquinones, asperuloside (rubichloric acid), damnacanthal, glycosides, morindadiol, morindine, morindone, mucilaginous matter, nor-damnacanthal, rubiadin, rubiadin monomethyl ether, resins, soranjidiol, sterols, and trihydroxymethyl anthraquinone-monomethyl ether; from the root bark: alizarin, chlororubin, glycosides (pentose, hexose), morindadiol, morindanigrine, morindine, morindone, resinous matter, rubiadin monomethyl ether, and soranjidiol; from the wood: anthragallol-2,3-dimethylether; from the tissue culture: damnacanthal, lucidin, lucidin-3-primeveroside, and morindone-6beta-primeveroside; from the plant: alizarin, alizarin-alpha-methyl ether, anthraquinones, asperuloside, hexanoic acid, morindadiol, morindone, morindogenin, octanoic acid, and ursolic acid.

Recently, many health benefits have been discovered stemming from the use of products containing Morinda citrifolia. One benefit of Morinda citrifolia is found in its ability to isolate and produce Xeronine, which is a relatively small alkaloid physiologically active within the body. Xeronine occurs in practically all healthy cells of plants, animals and microorganisms. Even though Morinda citrifolia has a negligible amount of free Xeronine, it contains appreciable amounts of the precursor of Xeronine, called Proxeronine. Further, Morinda citrifolia contains the inactive form of the enzyme Proxeronase which releases Xeronine from Proxeronine. A paper entitled, “The Pharmacologically Active Ingredient of Morinda citrifolia” by R. M. Heinicke of the University of Hawaii, indicates that Morinda citrifolia is “the best raw material to use for the isolation of xeronine,” because of the building blocks of Proxeronine and Proxeronase. These building blocks aid in the isolation and production of Xeronine within the body.

The function of the essential nutrient Xeronine is fourfold. First, Xeronine serves to activate dormant enzymes found in the small intestines. These enzymes are critical to efficient digestion, calm nerves, and overall physical and emotional energy. Second, Xeronine protects and keeps the shape and suppleness of protein molecules so that they may be able to pass through the cell walls and be used to form healthy tissue. Without these nutrients going into the cell, the cell cannot perform its job efficiently. Without Proxeronine to produce Xeronine our cells, and subsequently the body, suffer. Third, Xeronine assists in enlarging the membrane pores of the cells. This enlargement allows for larger chains of peptides (amino acids or proteins) to be admitted into the cell. If these chains are not used they become waste. Fourth, Xeronine, which is made from Proxeronine, assists in enlarging the pores to allow better absorption of nutrients.

Each tissue has cells which contain proteins which have receptor sites for the absorption of Xeronine. Certain of these proteins are the inert forms of enzymes which require absorbed Xeronine to become active. Thus Xeronine, by converting the body's procollagenase system into a specific protease, quickly and safely removes the dead tissue from skin. Other proteins become potential receptor sites for hormones after they react with Xeronine. Thus the action of Morinda citrifolia in making a person feel well is probably caused by Xeronine converting certain brain receptor proteins into active sites for the absorption of the endorphin, the well being hormones. Other proteins form pores through membranes in the intestines, the blood vessels and other body organs. Absorbing Xeronine on these proteins changes the shape of the pores and thus affects the passage of molecules through the membranes.

Because of its many benefits, Morinda citrifolia has been known to provide a number of anecdotal effects in individuals having cancer, arthritis, headaches, indigestion, malignancies, broken bones, high blood pressure, diabetes, pain, infection, asthma, toothaches, blemishes, immune system failure, and others.

In addition to the numerous health benefits, Morinda citrifolia also provides significant benefits to the skin. Morinda citrifolia is high in anti-oxidants that help to fight free-radical damage caused by the sun and other changing environmental conditions and elements. To stay healthy, the skin must cope with these elements and conditions and repair the damage caused at the same time. The skin is in a constant state of repair as it sheds the dead cells on the surface and replenishes the lower layers. Morinda citrifolia is also especially rich in linoleic acid, which is an essential fatty acid having the specific ability to nourish the health of the skin.

### Inhibiting Metastasis

As provided above, embodiments of the present invention relate to inhibiting metastasis of carcinogenic cells. In particular, embodiments of the present invention relates to medicinal products, as well as health and well-being food products, and particularly to a medicinal product or a health and well-being food product designed to inhibit, block, and/or prevent metastasis of carcinogenic cells away from a primary cancer site. The following provides a more detailed description of the Morinda citrifolia-based formulations and compositions used to treat metastasis and the methods used for administering these to a subject, including examples of experimental studies and the results attained.

Metastasis is the movement or spreading of malignant cancer cells from their original site to distant organs. Over 50% of cancer patients die from metastasis. In spite of a large amount of research focused in this area, there are very few drugs or therapeutic treatments available for patients with metastatic cancer disease and searching for new entities to conquer this deadly disease has been an urgent task for decades for both research scientists and medical doctors.

At least some embodiments of the present invention embrace the metastatic effect of Morinda citrifolia-PPT. As will be discussed below, studies on at least some embodiments include using the syngeneic tumor metastasis model of melanoma B16-F0 cells in C57B1/6J mice to identify that Morinda citrifolia-PPT is anti-metastatic. In an effort to find the possible mechanisms involved in the prevention, a study was performed of Morinda citrifolia-PPT by GPC (gel permeation chromatography) and its galacturonic acid content by UV spectrometer. Two In vitro tests were performed to examine possible pathways of Morinda citrifolia-PPT's anti-metastatic effect, inhibition of fibronectin adhesion and inhibition of endothelium tube formation. Based on experimental data and other research, we are able to suggest several likely mechanisms for Morinda citrifolia-PPT's anti-metastatic effect.

The following relates to representative methods and materials used to obtain the anti-metastatic effects. Those skilled in the art will appreciate that the following is representative of one or more embodiments of the present invention.

First, Morinda citrifolia-PPT was prepared. The yellow-greenish fruit of Morinda citrifolia was picked, covered, and placed on a screened table under the sun for approximately one week or until harvested. The harvested fruit was processed into a puree by a fruit processor, where the coarse residues and seeds were screened off. The derived puree was centrifuged to obtain the clear juice.

An equal volume of ethanol (99.9% purity, Sigma) was added to the juice and the content was mixed. The mixture was centrifuged and the residues were discarded. Another equal volume of ethanol (99.9% purity, Sigma) was added to the obtained liquid and mixed. The mixture was centrifuged and the precipitated residues were sustained and air dried. The dried residues were Morinda citrifolia-PPT.

An in vivo metastasis test was performed, wherein groups of 6 (positive control), 6 (Morinda citrifolia-PPT test group), and 9 (vehicle control) immunocompetent (6-8 weeks old), pathogen-free (SPF) C57BL/6 male mice were used in this study and bred in animal isolator (IVC racks) under specific pathogens free (SPF) condition at 23±1° C. Viable B16-F0 murine melanoma cells (ATCC CRL-6322, 6×10⁴ in 0.2 mL) were inoculated into experimental mice intravenously, via tail vein. Mice were administered the Morinda citrifolia-PPT, 1% in drinking water ad libitum, for one week prior to tumor cell implantation and three weeks afterwards. Additionally, 0.8 mg Morinda citrifolia-PPT/mouse was injected intraperitoneally (IP) for 21 consecutive days following tumor cell implantation. Concurrently, the reference agent, mitomycin, was administrated IP at 1 mg/Kg twice per week for six doses after tumor cell implantation.

In the vehicle control group, the distilled water was administrated at 10 ml/Kg IP for three consecutive weeks. The body weight for all mice was recorded at day 1, day 8, day 15, and day 22 following tumor cell implantation. The lungs were excised and fixed 21 days after tumor inoculation. Metastatic nodules on the lung surface were then counted under a dissecting microscope. The student's t test was used to determine the statistical difference between the groups of treatment and vehicle control groups in a statistic analysis.

An anion-exchange chromatography of Morinda citrifolia-PPT was performed, wherein ten grams of Morinda citrifolia-PPT were dissolved in 1 L of distilled water and filtered through 0.4 μM filter paper (Fisher Scientific). 200 μL of the prepared Morinda citrifolia-PPT solution was loaded to the anion-exchange column (HiLoad 26/10 Q Sepharose HP, Amersham Pharmacia Biotech) at 0.5 ml/min using a peristaltic pump (Amersham Pharmacia Biotech). Prior to applying the Morinda citrifolia-PPT solution, the column was generated by passing 5 column volumes of 1 N NaCl and distilled water. Five columns of distilled water and 0.1 N NaCl were sequentially passed through the column and the 0.1 N NaCl fraction was collected. The fraction was filtered through a PES-50 ultrafiltration membrane in a Millipore stirred cell (Fisher Scientific) to eliminate the Na⁺ Cl⁻ using 99.9% nitrogen gas under pressure. The filtrates were washed three times with distilled water before freeze-drying.

One assay measured the adhesion of NRK 2 (normal rat kidney cell, ATCC CRL-6509) cells to a fibronectin-coated well. Morinda citrifolia-PPT, the 0.1 N NaCl Morinda citrifolia-PPT fraction, GRGDSP peptide (SIGMA), and 0.4% DMSO (SIGMA, vehicle control) in modified MEM-HEPES buffer pH 7.4 (Hyclone) were incubated in separate wells for 30 minutes at 37° C. The reaction was initiated by addition of NRK 2 cells (2×10⁶/ml) and run for 30 minutes. Each well was then washed 6 times with Dulbecco's PBS (Hyclone) followed by addition of 5 μM calcein AM (SIGMA) and a further 2 hour incubation period. Quantitation of fluorescent intensity, resulting from interaction of calcein AM with cells attached to the fibronectin coated plate, was read by a SpectrFluor Plus plate reader (AG Flwith excitation at 485 nm and emission at 535 nm.

One milligram of Morinda citrifolia-PPT and the 0.1 N NaCl Morinda citrifolia-PPT fraction were dissolved in DDI water for GPC analysis. A calibration curve was made using Dextran (Dextran Standard Kit, Waters), molecular weight range 5,200 to 410,000. GPC conditions: Instrument—Waters Alliance separate module 2690 and 2410 refractive index detector; Column—Ultrahydrogel 500 and Ultrahydrogel linear 6-13 μM; 7.8×300 mm in series; Column temperature—40° C.; Mobile phase—0.1 M sodium nitrate. Flow rate—0.6 ml/min; Injection volume—50 μL; Refractive detector internal temperature—50° C.; The standard curve was fitted by Waters software (Empower, version 1.0) and the molecular weights of Morinda citrifolia-PPT and 0.1 N NaCl fraction were analyzed using this curve.

In a carbohydrate content analysis, 0.015 g of granulated Morinda citrifolia-PPT was weighed and spread evenly. 10.0 ml of distilled water was added in and shook vigorously until Morinda citrifolia-PPT was dissolved. 1.0 ml of the aliquot was measured into a tube and 3.5 ml chilled concentrated sulfuric acid was added. This solution was heated at 50° C. for 10 minutes, cooled to room temperature, transferred into a 10.0 ml volumetric flask, washed and diluted to the volume with distilled water. 0.6 ml of the solution was transferred into a chilled test-tube, 3.6 ml of chilled 0.0125 M of sodium tetraborate (SIGMA) reagent was added and mixed well. The content of the tube was heated in a boiling water bath for 10 minutes, then cooled to room temperature, and 60 μL of 0.15% m-Hydroxybiphenyl reagent was added. The content was mixed promptly and let set for 3 minutes and the absorbency was read at 520 nm by UV spectrometer (Varian). A standard curve was generated by using galacturonic acid (VWR Scientific) at concentrations of 0.05 mg/ml, 0.10 mg/ml, 0.15 mg/ml, and 0.2 mg/ml respectively.

Regarding angiogenesis, HUVECs (1.0×10⁴/well, ATCC CRL-1730) were placed in an earlier prepared 96-well matrigel. Morinda citrifolia-PPT was added at 1000 μg/ml, 100 μg/ml, 10 μg/ml, 1 μg/ml, and 0.1 μg/ml. Paclitaxel was used as positive control and added at 0.1 μM, 0.1 μM, 0.001 μM, 0.0001 μM, and 0.00001 μM, respectively. The plate was then incubated for 18-hours under an atmosphere of 5% CO₂ at 37° C. Morphology of the endothelial cell tubes, which resembled a capillary network, was evaluated by photo microscopy. Tube disruption was scored for each well. Minimum inhibitory concentration (MIC) can be determined and can be used to assess if Morinda citrifolia-PPT inhibited angiogensis. Each concentration was tested in duplicate.

The following tables and graphics illustrate the anti-metastatic effect of Morinda citrifolia-PPT:

TABLE 1
Effect of Morinda citrifolia-PPT on Experiment Metastasis of
B16-F0 Melanoma

| | | No. of Metastasis | |
|---|---|---|---|
| Treatment | N | Nodules in the Lung | % Inhibition |
| Distilled water at 10 ml/Kg | 1 | 55 | |
| | 2 | 51 | |
| | 3 | 40 | |
| | 4 | 64 | |
| | 5 | 54 | |
| | 6 | 49 | |
| | 7 | 45 | |
| | 8 | 63 | |
| | 9 | 80 | |
| X ± SEM^{a} | | 57 ± 4 | |
| Morinda citrifolia-PPT at | 1 | 46 | |
| 0.8 mg/mouse | | | |
| | 2 | 33 | |
| | 3 | 29 | |
| | 4 | 32 | |
| | 5 | 34 | |
| | 6 | 52 | |
| X ± SEM | | 38 ± 4 | 22%^{b} |
| Mitomycin at 1 mg/Kg | 1 | 23 | |
| | 2 | 24 | |
| | 3 | 19 | |
| | 4 | | |
| | 5 | | |
| | 6 | | |
| X ± SEM | | | 60%^{c} |

Relating to the Table 1 above, viable B16-F0 murine melanoma cells were inoculated into C57B1/6J mice. Mice were administered ad libitum 1% Morinda citrifolia-PPT one week prior to the implantation and throughout the experiment. 10 ml/mouse distilled water (vehicle control), 0.8 mg/mouse of Morinda citrifolia-PPT were injected I.P. into mice for 21 consecutive days following the inoculation. Mitomycin was used as a positive control and injected twice a week at 1 mg/Kg for a total of six doses. The lungs were excised and fixed at the end of experiment and the metastasis nodules on the lung surface were counted under a dissecting microscope. The test was used to determine the significant difference between the groups of treatment and vehicle control. (SEM=standard error of mean; ^{b}p<0.006; ^{c}p<0.005.)

TABLE 2
Body Weight of Post-Implantation (g), Mean ± SEM

| Treatment | N | Day1 | Day8 | Day15 | D22 |
|---|---|---|---|---|---|
| Distilled water | 1 | 25 | 24 | 25 | 25 |
| at 10 ml/Kg | | | | | |
| | 2 | 27 | 26 | 27 | 28 |
| | 3 | 28 | 28 | 27 | 27 |
| | 4 | 22 | 22 | 23 | 23 |
| | 5 | 21 | 22 | 23 | 24 |
| | 6 | 24 | 25 | 26 | 27 |
| | 7 | 21 | 22 | 24 | 25 |
| | 8 | 21 | 23 | 24 | 25 |
| | 9 | 23 | 24 | 25 | 25 |
| X ± SEM^{a} | | 24 ± 0.9 | 24 ± 0.7 | 25 ± 0.5 | 25 ± 0.5 |
| Morinda citrifolia- | 1 | 23 | 24 | 25 | 24 |
| PPT at | | | | | |
| 0.8 mg/mouse | | | | | |
| | 2 | 28 | 28 | 29 | 28 |
| | 3 | 25 | 25 | 28 | 27 |
| | 4 | 25 | 25 | 25 | 25 |
| | 5 | 26 | 26 | 27 | 26 |
| | 6 | 25 | 26 | 25 | 26 |
| X ± SEM | | 25 ± 0.7 | 26 ± 0.6^{a} | 27 ± 0.7^{b} | 26 ± 0.6 |
| Mitomycin at | 1 | 28 | 28 | 27 | 27 |
| 1 mg/Kg | | | | | |
| | 2 | 29 | 29 | 29 | 29 |
| | 3 | 26 | 27 | 26 | 26 |
| | 4 | | | | |
| | 5 | | | | |
| | 6 | | | | |
| X ± SEM | | | | | |

Relating to Table 2, mice body weight was recorded post-implantation. There was body weight increase in the Morinda citrifolia-PPT treated groups compared to the vehicle control group. (^{a}p<0.05; ^{b}p<0.05)

TABLE 3
Percentage adhesion inhibition, fibronectin mediated.

| | Morinda | | |
|---|---|---|---|
| | citrifolia- | 0.1 N NaCl | GRGDSP |
| Conc. (μg/ml) | PPT | Fraction | Peptide |
| 10,000 | 36 | 99 | |
| 1,000 | 19 | 37 | |
| 500 | | | 98 |
| 250 | | | 78 |
| 125 | | | 43 |
| 100 | 7 | 7 | |
| 62.5 | | | 22 |
| 31.25 | | | 16 |
| 10 | 8 | 1 | |
| 1 | 1 | 3 | |

Relating to Table 3, NRK 2 cells were used to adhere to fibronectin-coated wells. Quantitation of fluorescent intensity resulting from interaction of calcein AM with cells attached to the fibronectin coated plate was read by a SpectrFluor Plus plate reader.

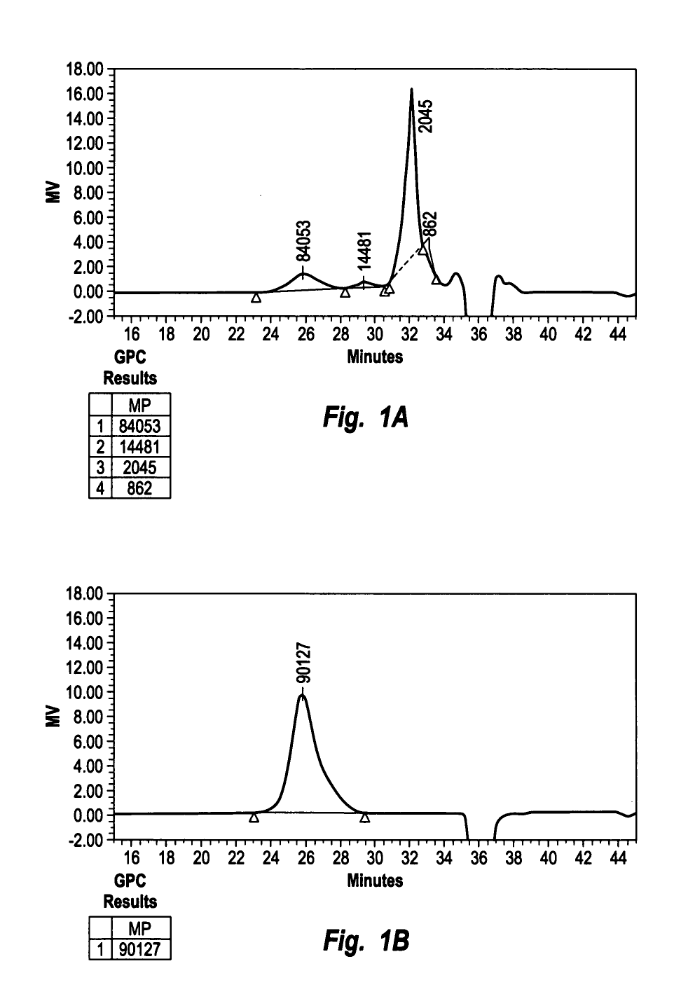

The following figures relate to chromatography, wherein FIG. 1A illustrates chromatography of Morinda citrifolia-PPT GPC analysis (The numbers represent molecular weight in Dalton for each corresponding peak.) and FIG. 1B illustrates chromatography of 0.1 M NaCl fraction GPC analysis.

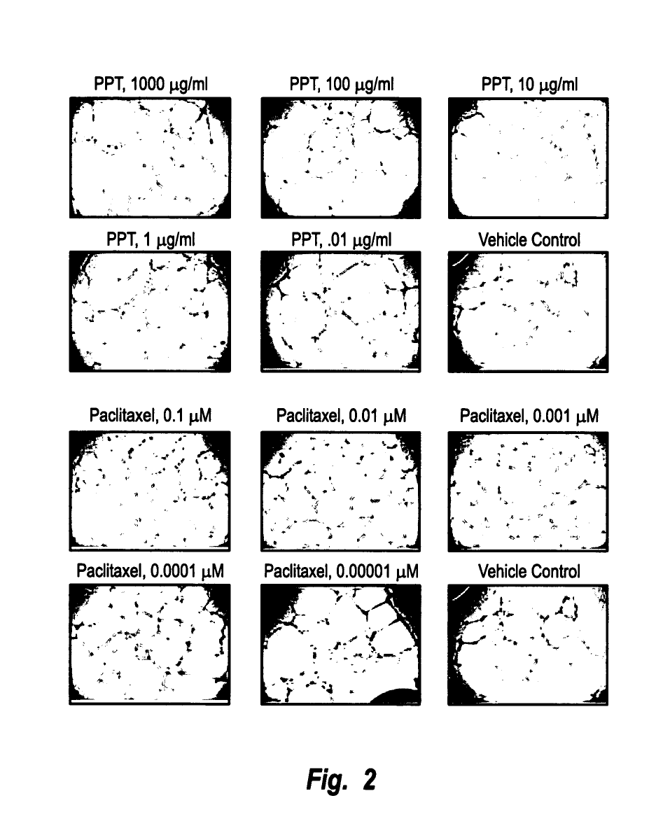

FIG. 2 illustrates photographs of tube formation for Morinda citrifolia-PPT, vehicle, and positive control, pacitaxel. As illustrated in FIG. 2, the Morinda citrifolia-PPT (as PPT) at tested concentration did not disrupt the continuous networks of the endothelial cell tubes. In the in vivo metastasis test, Morinda citrifolia-PPT demonstrated a metastatic inhibition effect using the synergic tumor metastasis model of melanoma B16-F0 cells in C57B1/6J mice. Combined with free drinking throughout the experiment, at 0.8 mg/mouse, Morinda citrifolia-PPT showed 22% inhibition (P<0.006). No increase in body weight was observed (See Tables 1-2). Chemotherapy drug, mitomycin, was used as a positive control and showed a 55% metastasis inhibition at 1 mg/Kg doses. There was no body weight increase in the positive control, as well.

Relating to adhesion, fibronectin mediated, Morinda citrifolia-PPT exhibited 36% inhibition, at 10 mg/ml, of NRK 2 cell adhesion to fibronection-coated wells. The 0.1 N NaCl fraction of Morinda citrifolia-PPT demonstrated a 37% adhesion inhibition, fibronectin mediated, at 1 mg/ml (See Table 3).

Regarding the GPC Analysis for components in Morinda citrifolia-PPT, by using gel permeation chromatography (GPC), four major peaks were found and revealed that Morinda citrifolia-PPT was composed of four different molecular weight molecules at MW 862, MW 2,045, MW 14,461, and MW 84,076 Dalton respectively (see FIG. 1A). The 0.1 N NaCl fraction was analyzed by GPC and found to have a molecular weight at 90,127 Dalton (see FIG. 1B). This corresponded to the peak of MW 84,058 Dalton in Morinda citrifolia-PPT, and the molecular weight shift was due to experimental error. Area analysis showed that the peak of MW 84,058 had a 12.5% of the total area of the derived four peaks.

Regarding a carbohydrate content analysis, the carbohydrate analysis demonstrated that Morinda citrifolia-PPT contained 70% galacturonic acid determined by UV spectrometer.

Regarding an angiogenesis, in vitro tube formation assay, there was no tube disruption observed for Morinda citrifolia-PPT at all concentrations tested (See FIG. 2).

In the study discussed above, it was demonstrated that Morinda citrifolia-PPT was anti-metastatic against the melanoma B16-F0 cell in C57B1/6J mice. The anti-metastatic effect of Morinda citrifolia-PPT involved the prevention of tumor cell adhesion to fibronectin, as indicated by the 36% adhesion inhibition of Morinda citrifolia-PPT at 10 mg/ml in the test. Fibronectin is a large adhesive glycoprotein found in extracellular matrices and body fluids. The fibronectin-specific intergrin, which consists of a α₅ and a β₁ subunit, is the major fibronectin receptor in most cells. This integrin binds to the RGD site of fibronectin and mediates celluar response to binding, such as adhesion, migration, organization of a cytoskeleton and assembly of the fibronectin extracellular matrix. Studies show that anti-β₁ and anti-α₅ antibodies inhibited cell migration on fibronectin for several carcinoma cells, including HT-1080 fibrosarcoma, 5637 bladder carcinoma, and MDA-231 breast carcinoma. The PHSRN site of fibronectin, not the RGD site, induced the invasion of the rat prostate metastatic carcinoma cell, MLL. This finding was consistent with other studies in that the PHSRN site was defined as the synergy site of RGD because only when both sites co-existed in relative orientation, the substantial activity of cell adhesion could be achieved.

In the experiment discussed above, Morinda citrifolia-PPT exhibited a 36% inhibition of fibronectin mediated adhesion at 10 mg/ml. The 0.1 N NaCl fraction of Morinda citrifolia-PPT, MW 84,076 Da, exhibited a 37% adhesion inhibition activity at 1 mg/ml. Considering that this fraction was about 12.5% of Morinda citrifolia-PPT, it contributed almost entirely to the total anti-adhesion activity. However, the relatively weak activity of Morinda citrifolia-PPT also suggested that there are other mechanisms of the anti-metastasis effect involved.

Another mechanism proposed here is based on the theory that certain cell types prefer to metastasize to specific target organs and the observation that Morinda citrifolia-PPT is composed of 70% galacturonic acid. More and more evidence indicated that the selection of a target organ for metastasis is mediated by specific interactions between carcinoma cells and the endothelium cells of that target organ. Such interactions involve cell surface molecules, which are comprised of constitutively expressed membrane glycoproteins that have restricted distributions in the blood vessels of one or more organs. An endothelial surface molecule, lung-derived endothelial cell adhesion molecule-1 (Lu-ECAM-1), was isolated and found to mediate and arrest lung metastasis of melanoma cells. Syngeneic mice immunized with anti-Lu-ECAM-1 mAb 6D3 1 h prior to an intravenous injection of B16-F10 cells showed more than 90% reduction in the number of lung colonies compared to the control. Analysis of Lu-ECAM-1 structure indicated that it was a glycoprotein, with sugar content at 3%. A series of carbohydrates were tested for adhesion inhibition of B16-F10 to Lu-ECAM-1. Results demonstrated that Lacto-N-fucopentose I most effectively blocked adhesion B16-F10 to Lu-ECAM-1. This finding implied that Lu-ECAM-1 was a galacoside-binding protein.

In our sugar analysis, we found that Morinda citrifolia-PPT contained 70% galacturonic acid. It is, therefore, possible that Morinda citrifolia-PPT exherts its anti-metastatic effect by binding to Lu-ECAM-1 molecules and blocked adhesion melanoma B 16-F0 cell to Lu-ECAM-1. Similar proteins in the same family as Lu-ECAM-1 have been discovered, including hCLCA3 (Gruber et al., 1999). hCLCA3 is expressed in numerous tissues such as lung, trachea, spleen, thymus, and mammary gland indicating the possible anti-metastatic effect of Morinda citrifolia-PPT on these tissues.

The galacturonic acid content of Morinda citrifolia-PPT suggests its anti-metastasis effect involves inhibiting cell-cell and cell-substrate binding. Many malignant cells have the ability to bind exogenous carbohydrate-containing-ligands. Galectin-galactoside binding protein-expression has been found in human melanoma, colorectal, gastric, and papillary thyroid carcinomas. These cell surface galectins induced cell aggregation and cell attachment to the substratum and play a key role in tumor cell invasion. Cell aggregation and attachment were found to be suppressed by binding of monoclonal anti-galectin antibodies to the cell surface. Simple sugars, such as D-galactose and arabinogalctose, inhibit liver metastasis of L-1 sarcoma cells. A galacturonic acid rich modified citrus pectin was demonstrated to inhibit lung metastasis of rat MAT-LyLu prostate tumor cells in male Copenhagen rats. In the same study, the expression of galectin-3 in rat MAT-LyLu cells found that a concentration correlation between the metastasis inhibition in vivo and the adhesion inhibition in vitro.

Since Morinda citrifolia-PPT has no effect on B 16-F0 tumor growth (no body weight increase observed during the experiment time) and blood vessel endothelial cell growth (see result), it seems that its anti-metastatic effect is by acting as an anti-adhesive agent, inhibiting the formation of tumor cell emboli and the tumor cell-cell interaction.

Accordingly, as can be seen from the studies and from the in vivo and in vitro tests, there is an inhibitoratory effect of Morinda citrifolia-PPT and this anti-metastatic effect results in a reduction in blood vessels and tumor metastasis.

As provided above, the previous discussion related to one or more representative embodiments in accordance with the present invention. Those skilled in the art will appreciate that the present invention embraces other embodiments that relate to inhibiting metastasis of carcinogenic cells. Accordingly, the following relates to additional embodiments of the present invention:

| | |
|---|---|
| Ingredients | Percent by Weight |

Formulation One

| | |
|---|---|
| Morinda citrifolia Fruit Juice | 100% |

Formulation Two

| | |
|---|---|
| Morinda citrifolia Fruit Juice | 85-99.99% |
| Water | 0.1-15% |

Formulation Three

| | |
|---|---|
| Morinda citrifolia Fruit Juice | 85-99.99% |
| Other fruit juices | 0.1-15% |

Formulation Four

| | |
|---|---|
| Morinda citrifolia Fruit Juice | 50-90% |
| Water | 0.1-50% |
| Other fruit juices | 0.1-30% |

In one embodiment, a person suffering from metastasis as described above takes at least one (1) ounce of Formulation One in the morning on an empty stomach, and at least one (1) ounce at night on an empty stomach, just prior to retiring to bed. In one example, which is not meant to be limiting in any way, the beneficial Morinda citrifolia is processed into Tahitian Morinda citrifolia® juice manufactured by Morinda, Incorporated of Orem, Utah.

In another preferred embodiment, a person experiencing metastasis takes at least one ounce of Formulation Two twice a day until the overgrowth is abated.

The following examples set forth and present the effects of Morinda citrifolia on the metastasis of carcinogenic cells. These examples are not intended to be limiting in any way, but are merely illustrative of the beneficial effects of Morinda citrifolia as processed and manufactured herein. Other non-limiting examples of the present invention are described below.

### EXAMPLE ONE

In the present example, a patient is experiencing the metastasis of carcinogenic cells from the primary or initial cancer location or site. The individual suffering from the metastasis desires to treat the condition with a nonprescription, over-the-counter preparation. To treat the infection, the individual consumes a prescribed amount of food product composition containing processed Morinda citrifolia fruit juice. The person intermittently consumes the food product containing the processed Morinda citrifolia fruit juice until the metastasis is inhibited, blocked, and/or prevented or the metastasized cells are destroyed and the infection is reduced or eliminated.

### EXAMPLE TWO

The present example presents the effects of Morinda citrifolia, coded as MDA-4 or PPT, on metastasis of carcinogenic cells. Specifically, the following example presents the anti-metastasis activity of Morinda citrifolia (MDA-4) on the syngeneic tumor model of murine melanoma B16-F0 cells in C57BL/6J mice.

In the syngeneic tumor metastasis model of melanoma B16-F0 cells in C57BL/6J mice, MDA-4 was administered at 1% (10 mg/ml) by a drinking method for one week for pre-treatment, and for three weeks after the tumor cells were implanted, during the study and throughout the experiment for a total of 28 treatments. Additionally, MDA-4 at a dose of 0.8 mg/mouse was administered intraperitoneally (IP) daily for a total of 21 consecutive days after tumor cells were implanted in the mice.

In measuring tumor metastatic nodules as the metastasis index, the data of nodules in the lungs relative to the vehicle control group for animals treated with MDA-4 are shown in Table 4.

TABLE 4

| | | | | No. of | |
|---|---|---|---|---|---|
| | | | | Metastastic | |
| | | | | Nodules in | |
| Treatment | Route | Dose | N | the Lung | % Inh. |
| Vehicle | IP | 10 ml/kg × 21 | 3 | 49 ± 4 | — |
| (D.W.) | | | | | |
| MDA-4 | Drinking | 1% × 28 & | 6 | 38 ± 4 | 22 |
| | & IP | 0.8 mg/mouse × 21 | | | |
| Mitomycia | IP | 1 mg/kg × 6 | 3 | 22 ± 2** | 55 |

The test is used to determine significant difference between test substance treated and vehicle control. (Groups (**P<0.01))

Based on the results obtained, MDA-4 at a dose of 0.8 mg/mouse by intraperitoneal injection, combined with the free drinking method of 1% MDA-4, exhibited a slight inhibition of tumor metastasis. (See Table 5). Body weight was also monitored during the study and no significant difference in the change was observed when compared with the vehicle control group. (See Table 6.) During the experiment, it was noted that the animals showed no toxic symptoms after the test compound administration.

During the experiment, the test substance MDA-4 was provided by Morinda, Inc. and used for in vivo anti-metastasis studies. MDA-4 was dissolved in sterile distilled water and, as stated, administrated with 1% (10 mg/ml) drinking method for one week pre-treatment, and three week after tumor cells were implanted during the study throughout the experiment for a total of 28 treatments. Additionally, as stated, MDA-4 at dose of 0.8 mg/mouse was administrated intraperitoneally (IP) daily for a total of 21 consecutive days after tumor cells implanted.

The murine melanoma cell line, B16-F0 (ATCC CRL-6322), was purchased from American Type Culture Collection and Dulbecco's Modified Eale's medium, 90% with Fetal Bovine Serum, 10% was used as culture medium. The tumor cells were incubated in an atmosphere containing 5% CO₂ at 37° C. The medium was supplemented with 1% Antibiotic-Antimycotic.

Specific pathogen-free male C57BL/6J mice at 6-8 weeks of age, weighing 24-28 gms were provided by National Taiwan University Animal Center. The animals were housed in Individually Ventilated Cages Racks (IVC Racks, 36 Mini Isolator System) under Specific Pathogen-Free (SPF) conditions throughout the experiment.

Each APEC® cage was autoclave sterilized and contained 3 mice (in cm, 26.7 length×20.7 width×14.0 height), and then maintained in a hygienic environment under controlled temperature (22° C.-24° C.) and humidity (60%-80%) with 12 hours light/dark cycle. The animals were given free access to sterilized distilled water ad libitum. All aspects of this work, i.e., housing, experimentation and disposal of animals was performed in general according to the International Guiding Principles for Biomedical Research Involving Animals (CIOMS publication No. ISBN 9290360194, 1985).

The chemicals used were Antibiotic-Antimycotic (GIBCO BRL, USA), Fetal Bovine Serum (HyClone, USA), Dulbecco's Modified Eagle's medium (HyClone, USA) and Mitomycin (Kyowa, Japan).

Some of the equipment used comprised Centrifuge 5810R (eppendorf, Germany), CO₂ Incubator (Form a Scientific Inc., USA), Hemacytometer (Hausser Scientific Horsham, USA), Individually Ventilated Cages Racks (IVC Racks, 36 Mini Isolator system) (Techniplast, Italy), Inverted Microscope CK-40 (Olympus, Japan), System Microscope E-400 (Nikon, Japan) and Vertical laminar flow (Tsao-Hsin, R.O.C.).

According to the preferred method of evaluation, groups of three or six (6-8 weeks old) specific pathogen-free (SPF) C57BL/6J male mice bred in an animal isolator (IVC racks) under SPF condition at 23±1° C. were used. Viable B16-F0 murine melanoma cells (ATCC CRL-6322, 6x10r in 0.2 ml) were inoculated into experimental mice intravenously via the tail vein.

A 1% of test compound MDA-4 was given to the animals by a drinking method and treatment started one week prior to tumor inoculations (as day 0) and continuously treated daily administration until day 21 for a total of 21 consecutive days after the tumor cells were implanted. Concurrently, the reference agent mitomycin was administrated intraperitoneally twice a week for six doses after tumor cells implantation. The lungs were excised and fixed 21 days after tumor inoculation. Metastasis nodules on the lung surface were then counted under a dissecting microscope. The following results were obtained:

TABLE 5
Effect of Morinda citrifolia on Experiment Metastasis of B16-F0 Melanoma

| | | | | No. of Metastastic Nodules in the Lung | | | | |
|---|---|---|---|---|---|---|---|---|
| | | | | | | | Total | % |
| Treatment | Route | Dose | N | R < 1 mm | 1 mm< | R > 2 mm | Nodules | Inhibition |
| Vehicle | IP | 10/ml kg × 21 | 1 | 30 | 15 | 10 | 55 | |
| Control | | | 2 | 30 | 14 | 7 | 51 | |
| (D.W.) | | | 3 | 25 | 10 | 5 | 40 | |
| | | | X ± SEM | | | | 49 ± 4 | |
| PT#1022457 | Drinking | 1% × 28 & | 1 | 31 | 13 | 2 | 46 | |
| (MDA-4) | & IP | 0.8 mg/ | 2 | 21 | 11 | 1 | 33 | |
| (PPT) | | mouse × 21 | 3 | 15 | 8 | 6 | 29 | |
| | | | 4 | 17 | 13 | 2 | 32 | |
| | | | 5 | 17 | 11 | 6 | 34 | |
| | | | 6 | 32 | 14 | 6 | 52 | |
| | | | X ± SEM | | | | 38 ± 4 | 22 |
| Mitomycin | IP | 1 mg/kg × 6 | 1 | 16 | 6 | 1 | 23 | |
| | | | 2 | 17 | 7 | 0 | 24 | |
| | | | 3 | 15 | 4 | 0 | 19 | |
| | | | X ± SEM | | | | 22 ± 2** | 55 |

In this example or table of results, R refers to the diameter of a metastatic tumor nodule and are scored as follows: a=the number of nodules with (R<1 mm), b=the number of nodules with (1 mm≦R<2 mm), c=the number of nodules with (R≧2 mm). The student's t test is used to determine significant difference between test substance treated and vehicle control groups.

TABLE 6
Effect of Morinda citrifolia on Experiment Metastasis of B16-F0 Melanoma

| | | | | Body Weight of Post-Implantation (g), | | | |
|---|---|---|---|---|---|---|---|
| | | | | Mean ± SEM | | | |
| Treatment | Route | Dose | N | Day 1 | Day 8 | Day 15 | Day 22 |
| Vehicle | IP | 10/ml kg × 21 | 1 | 25 | 24 | 25 | 25 |
| Control | | | 2 | 27 | 26 | 27 | 28 |
| (D.W.) | | | 3 | 28 | 28 | 27 | 27 |
| | | | | 26.7 ± 0.9 | 26 ± 1.2 | 26.3 ± 0.7 | 26.7 ± 0.9 |
| PT#1022457 | Drinking | 1% × 28 & | 1 | 23 | 24 | 25 | 24 |
| (MDA-4) | & IP | 0.8 mg/ | 2 | 28 | 28 | 29 | 28 |
| (PPT) | | mouse × 21 | 3 | 25 | 25 | 28 | 27 |
| | | | 4 | 25 | 25 | 25 | 25 |
| | | | 5 | 26 | 26 | 27 | 26 |
| | | | 6 | 25 | 26 | 25 | 26 |
| | | | | 25.3 ± 0.7 | 25.7 ± 0.6 | 26.5 ± 0.7 | 26.0 ± 0.6 |
| Mitomycin | IP | 1 mg/kg × 6 | 1 | 28 | 28 | 27 | 27 |
| | | | 2 | 29 | 29 | 29 | 29 |
| | | | 3 | 26 | 27 | 26 | 26 |
| | | | | 27.7 ± 0.9 | 28.0 ± 0.6 | 27.3 ± 0.9 | 27.3 ± 0.9 |

### EXAMPLE THREE

This study focuses on the inhibition of tumor metastasis of an ethanol insoluble precipitate from Morinda citrifolia fruit juice, and presents or sets forth one possible mechanism. In this study, an ethanol insoluble precipitate (polysaccharides) from Morinda citrifolia fruit of Tahiti, French Polynesia, Morinda citrifolia-PPT, was found to have anti-metastatic activity in the syngeneic tumor metastasis model of melanoma B16-F0 cells in C57B1/6J mice. Morinda citrifolia-PPT showed 22% inhibitory effect (p<0.005) at 0.8 mg/mouse. The possible mechanisms of Morinda citrifolia-PPT's anti-metastatic effect were investigated.

Gel Permeation Chromatography (GPC) analysis demonstrated that Morinda citrifolia-PPT is composed of four different molecular weight molecules, at 879, 2045, 14,461, and 84,076 Daltons, respectively. Using anion exchange chromatography, the fraction with MW 84,076 was derived by eluting Morinda citrifolia-PPT with 0.1 N NaCl. Morinda citrifolia-PPT and the fraction with MW 84,076 showed 36% and 37% adhesion inhibition at 10 mg/ml and 1 mg/ml, respectively, of NRK 2 cells to a fibronectin-coated well. Although the inhibition of fibronectin adhesion might be one of the possible mechanisms for Morinda citrifolia-PPT's anti-metastatic effect, the relatively weak adhesion effect in the fibronectin mediated adhesion experiment suggested that there might be more than one mechanism involved. The carbohydrate content analysis indicated that Morinda citrifolia-PPT contained 70% galacturonic acid, determined by UV spectrometer.

This finding implied that Morinda citrifolia-PPT may suppress the adhesion of melanoma B16-F0 to the lung tissue by binding to a lung endothelial surface molecule, Lu-ECAM-1-a galacoside-binding protein. It also suggested that Morinda citrifolia-PPT may exert anti-metastasis effects through blocking cell-cell and cell-substrate binding for many malignant cell surface expressed galectin-galactoside binding proteins. Morinda citrifolia-PPT did not exhibit any inhibition effect of endothelial cell tube formation. It was therefore, that Morinda citrifolia-PPT's anti-metastatic effect is more likely by acting as an anti-adhesive agent to inhibit the formation of tumor cell emboli and the tumor cell-cell interaction. The details of the study are set forth herein.

Thus, as discussed herein, the embodiments of the present invention embrace inhibiting metastasis of carcinogenic cells. In particular, the present invention relates to medicinal products, as well as health and well-being food products, and particularly to a medicinal product or a health and well-being food product designed to inhibit, block, and/or prevent metastasis of carcinogenic cells away from a primary cancer site.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

1. A naturaceutical formulation for treating, inhibiting, and preventing metastasis of carcinogenic cells, said naturaceutical formulation comprising:
processed Morinda citrifolia product present in an amount between about 0.01 and 100 percent by weight.

2. A naturaceutical formulation as recited in claim 1, wherein the processed Morinda citrifolia is processed Morinda citrifolia juice.

3. A naturaceutical formulation as recited in claim 2, further comprising water.

4. A naturaceutical formulation as recited in claim 3, wherein the Morinda citrifolia juice is present in an amount between about 85% to 99.99% by weight.

5. A naturaceutical formulation as recited in claim 3, wherein the Morinda citrifolia juice is present in an amount between about 50% to 90% by weight.

6. A naturaceutical formulation as recited in claim 2, further comprising another fruit juice.

7. A naturaceutical formulation as recited in claim 1, wherein the Morinda citrifolia is Morinda citrifolia-PPT.

8. A method for inhibiting and preventing metastasis of carcinogenic cells, as well as destroying early stage metastasized carcinogenic cells, said method comprising the steps of:
adding a processed Morinda citrifolia product to an alcohol-based solution;
isolating and extracting an active ingredient of Morinda citrifolia from said solution; and
introducing said extracted active ingredient to an area afflicted by said carcinogenic cells, wherein said extracted active ingredient inhibits and prevents further growth of said carcinogenic cells, as well as destroys early stage metastasized carcinogenic cells.

9. The method of claim 8, wherein said processed Morinda citrifolia product comprises processed Morinda citrifolia fruit juice.

10. The method of claim 8, wherein said processed Morinda citrifolia product comprises processed Morinda citrifolia puree.

11. The method of claim 8, wherein said processed Morinda citrifolia product comprises processed Morinda citrifolia-PPT.

12. The method of claim 11, wherein said alcohol-based solution is selected from the group consisting essentially of methanol, ethanol, and ethyl acetate, and other alcohol-based derivatives.

13. The method of claim 12, wherein said active ingredient is Quercetin.

14. The method of claim 13, wherein said active ingredient is Rutin that synergistically works with said Quercetin to inhibit and prevent metastasis of said carcinogenic cells.

15. A method for inhibiting, preventing, and destroying further growth of metastasized cancer cells, said method comprising the steps of:
orally administering at least one ounce of a naturaceutical formulation comprising processed Morinda citrifolia fruit juice on an empty stomach in the morning; and
orally administering at least one ounce of said naturaceutical formulation prior to sleeping at night.

16. The method of claim 15, wherein the Morinda citrifolia comprises Morinda citrifolia-PPT.

17. The method of claim 16, wherein said naturaceutical formulation comprises the Morinda citrifolia-PPT present in an amount by weight of up to 100 percent.

18. The method of claim 14, wherein said naturaceutical formulation comprises:
processed Morinda citrifolia fruit juice present in an amount by weight between about 85-99.99 percent; and
water present in an amount by weight between about 0.1-15 percent.

19. The method of claim 14, wherein said naturaceutical formulation comprises:
processed Morinda citrifolia fruit juice present in an amount by weight between about 85-99.99 percent; and
other fruit juices present in an amount by weight between about 0.1-15 percent.

20. The method of claim 14, wherein said naturaceutical formulation is consumed by orally administering 2 ounces of said food product, twice daily.
